(19) United States
(12) Patent Application Publication
(10) Pub. No.: US 2022/0152391 A1
(43) Pub. Date: May 19, 2022

(54) METHOD OF INCREASING THORACIC ROTATION RANGE OF MOTION

(71) Applicant: Gene Jason Hunt, Lutz, FL (US)

(72) Inventor: Gene Jason Hunt, Lutz, FL (US)

(21) Appl. No.: 16/949,884

(22) Filed: Nov. 19, 2020

(51) Int. Cl. A61N 1/36

(52) U.S. Cl. CPC A61N 1/36031; A61B 5/1121; A61N 1/36034; A61N 1/36003

(57) ABSTRACT

A method for treating deficiencies of a patient's thoracic rotation. The method includes comparing the symmetry of the range of motion for the thoracic rotation, lumbar rotation, thoracic flexion, and/or lumbar flexion about the spine using a motion capture system. The method further includes providing electrical simulation to a sequence of muscles associated with the thoracic rotation, lumbar rotation, thoracic flexion, and/or lumbar flexion for increasing the symmetry by elongating relevant muscles of the sequence of muscles.

### BACKGROUND OF THE INVENTION

The present invention relates to methods of treating limited range of motion for thoracic rotation and, more particularly, a method embodying the use of three-dimensional (3D) motion capture of the spine for relevant data capture that is integrated into a practical application of using electrical stimulation to increase thoracic rotation in a patient.

The loss of range of motion in the thoracic spine puts undue stress on the lumbar spine and hips. This can be acutely debilitating for golfers who need a robust range of motion in their thoracic rotation to generate power and control of their golf strokes. Specifically, regarding golf, the greater range of thoracic rotation the faster the golfer swings the golf club, the greater the force that is translated to the golf ball and, thus, generating more speed and power. Conversely, if the upper body's rotation is limited in range of motion relative to the spine, the less speed and power that the body can generate.

Historically, physical therapists or sports performance professionals have prescribed a long course of exercise and stretching to increase thoracic range of motion. This takes months or years to produce meaningful results. Furthermore, recent research shows that the effects of stretching does not have lasting effects on thoracic mobility.

Recent research, however, does show that the term X-factor is used to describe the rotation of the shoulders in relation to the hip throughout the golf swing. The research has also found different results from investigating the X-factor. For instance, rather than the X-factor at the top of the backswing, it was found that the X-factor after the initiation of the downswing was what differentiated between highly skilled (handicap less than 0 and one long drive champion) and less skilled golfers (handicap 15+).

This phenomenon is known as X-factor stretch, which is the increase in shoulder and pelvic separation at the initiation of the downswing, which is achieved by the downswing starting with a rotation of the hips towards the target, independent of the shoulders.

The T-spine, which stands for thoracic spine, is that area of the body encompassing the chest and middle of the back. In most golf fitness discussions, however, the main reference is to the mid-back. The primary muscle groups include the lats, trapezius, and rhomboids.

Mobility in the T-spine is imperative if one wants to make a good upper body turn in both the back and downswing phases. It is also essential if you want to reduce the potential for lower back injury.

Limited range of motion in the upper body will more than likely result in a limited backswing, which decreases the distance the clubhead travels and subsequently, the ability to generate clubhead speed.

A tight mid-back may also result in an excessive hip turn as the golfer with limited ability to disassociate the upper body from the lower, forces the rotation of the body pulling the hips around along with the shoulders. A smaller X-factor angle equates to less power. Tightness in the mid-back can also lead up to the reverse spine swing fault if the golfer, lacking range of motion, tries to force the club back further in the backswing. This is especially true if the tightness occurs on the target side of the back (left side for a right-handed golfer). Trying to rotate beyond the stretch threshold will pull the upper body back toward the target, leaving the golfer in a poor hitting position at the top of the backswing. This usually results in an over-the-top downswing and the dreaded consequences.

A very real second concern of a tight mid-back is low back pain. There is a pattern of movement in the body that needs to be addressed in order to produce efficient movement. It starts in the foot and alternates between stability and mobility as you move up the body. For example, the foot needs to be stable, the ankle mobile, the knee stable, the hip mobile, and so on up the chain. In this pattern of movement, the lower back should be stable and the T-spine mobile. If a golfer is lacking in range of motion in the T-spine, the lower back will more than likely be asked to pick up the slack and assist more in the rotation of the upper body.

This recruitment of the lumbar spine places undue torque and stress to the lower back and sets it up for both acute and chronic injury. Improved thoracic rotation will create better separation and relieve pressure from lumbar spine and hips. This applies to every golfer who uses the modern swing.

As can be seen, there is a need for a method embodying the use of three-dimensional (3D) motion capture of the spine for relevant data capture that is integrated into a practical application of using electrical stimulation to produce a demonstrative same-day increase in thoracic rotation range of motion in the patient.

Using a 3D motion capture system (e.g., DARI) enables access to the patient's range of motion in the thoracic spine within 1/10 of a degree; thereby, restrictions and/or deficiencies of thoracic spine range of motion (such as asymmetrical motion) are identifiable by way of applying DC current electrical stimulation though the systemic protocols. The systemic protocols are based on subject findings on the 3D motion capture output. Specifically, thoracic rotation is affected by the internal and external oblique muscles. Therefore, the present invention includes a method embodying the placement the pads of the DC electrical stimulation on the affected muscles to selectively elongate the affected muscle groups, in order to realize an increase thoracic range of motion in the spine and so restore an overall more symmetrical range of motion. Once the muscles are elongated and balanced range of motion is restored, the method includes rescreening the patient with the 3D motion capture to confirm increased range of motion and symmetry. This leads to less undue stress on the lumbar spine and hip, which in turn will diminish the number of lumbar spine injuries in patients frequently using their thoracic rotation range of motion, such as golfers during their golf swings.

### SUMMARY OF THE INVENTION

In one aspect of the present invention, a method of increase a range of motion for thoracic rotation about a spine of a patient, the method includes the following: determining whether the patient has one or more thoracic range of motion deficiencies by: obtaining said range of motion relative within a tenth of a degree; and performing or having performed a pre-treatment report comparing a symmetry of thoracic rotation about a spine; and if the patient has said one or more thoracic range of motion deficiencies, then externally administering an electrical stimulation for less than infinity to the patient along a sequence of muscles operatively associated with said symmetry.

In another aspect of the present invention, the method further includes the following: performing or having performed a post-treatment report comparing the symmetry of thoracic rotation about the spine; and if the patient has said one or more thoracic range of motion deficiencies after the post-treatment report, then externally administering the electrical stimulation to the patient along said sequence of muscles, wherein the electrical stimulation endures for less than thirty minutes, wherein the pre-treatment report further compares a symmetry of lumbar rotation, thoracic flexion, and lumbar flexion about said spine, wherein the electric simulation elongates said sequence of muscles, whereby said symmetry is increased.

These and other features, aspects and advantages of the present invention will become better understood with reference to the following drawings, description and claims.

### BRIEF DESCRIPTION OF THE DRAWINGS

FIG. 1 is a schematic view of an exemplary embodiment of the present invention; and

FIG. 2 is a flow chart of an exemplary embodiment of the present invention.

### DETAILED DESCRIPTION OF THE INVENTION

The following detailed description is of the best currently contemplated modes of carrying out exemplary embodiments of the invention. The description is not to be taken in a limiting sense, but is made merely for the purpose of illustrating the general principles of the invention, since the scope of the invention is best defined by the appended claims.

Broadly, an embodiment of the present invention provides a method providing treatment for deficiencies of a patient's thoracic rotation. The method includes comparing the symmetry of the range of motion for the thoracic rotation, lumbar rotation, thoracic flexion, and/or lumbar flexion about the spine using a three-dimensional motion capture system. The method further includes providing electrical simulation to a sequence of muscles associated with the thoracic rotation, lumbar rotation, thoracic flexion, and/or lumbar flexion for increasing the symmetry by elongating relevant muscles of the sequence of muscles.

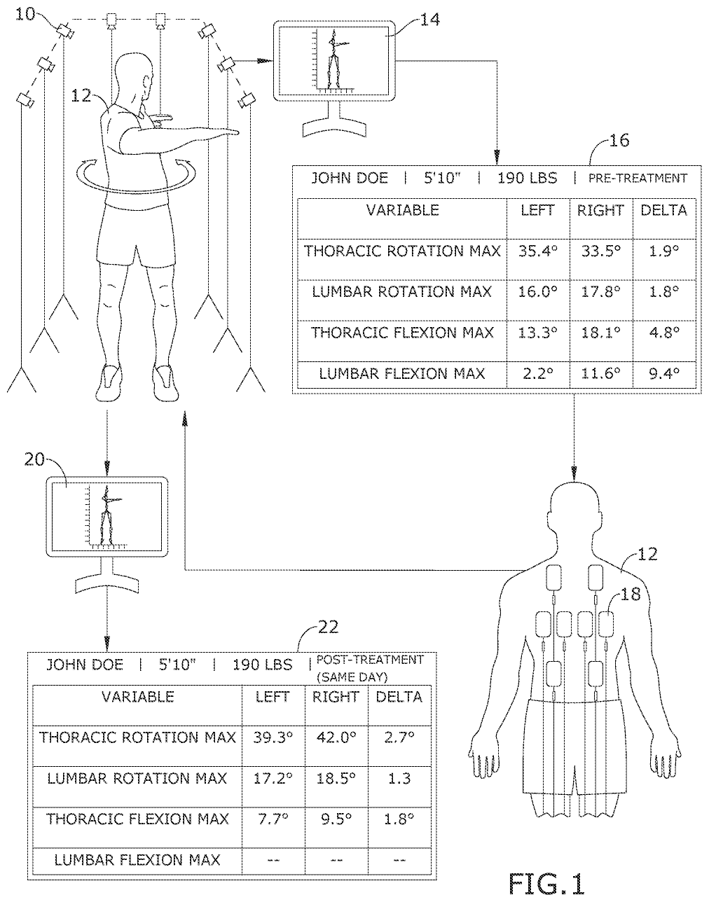

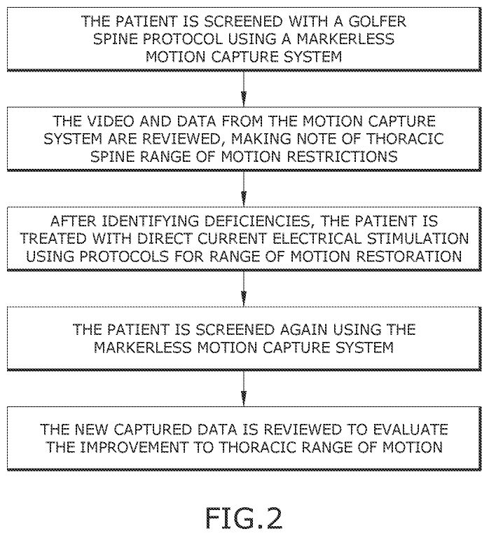

Referring now to FIGS. 1 and 2, the present invention may include the following systemic components. First, a trained practitioner would screen a patient 12 according to a golfer spine protocol using a marker-less motion capture system 10. Golfer spine protocol uses measurement of flexion of the thoracic spine and flexion of the lumbar spine, followed by unrestricted trunk rotation, reverse lunge combined with right and left rotation, hip abduction, and balance sway measurement.

After review of the video and initial range of motion data 14 captured by the motion capture system 10 during an initial screening, the practitioner takes note of range of motion restrictions of the thoracic spine. Then the practitioner may create a pre-treatment data report 16, and then treat patient with direct current electrical stimulation device, by way of electrodes 18, with the above-mentioned protocols designed to restore range of motion deficits From there, the practitioner rescreens 20 the patient subject on the motion capture system 10 to evaluate improvement of thoracic range of motion, and then generates a post-treatment data report 22.

The steps of the invention must be followed in order. There must be accurate data with the motion capture system. Once that data is collected it is essential to interpret the data properly. Specifically, a medical practitioner may use the data to make a treatment plan that addresses the deficits found on the screening process. For example if subject is lacking thoracic range of motion, electrical stimulation pads are placed on internal and external oblique muscles and the subject is taken through a progression of increased intensity of electrical stimulation coupled with active range of motion movements that increase thoracic rotation. Once the range of motion deficient is identified, it is essential to understand how the human body works, what muscle groups are involved the restriction and normal rotational function of the thoracic spine. It is then essential to know how to use the electrical stimulation device to produce desired outcomes, including elongating the proper sequence of muscles—for instance, elongation and symmetry of internal and external oblique muscles along with smaller spinal muscles—to allow for restoration of the range of motion. A deep understanding of the golf swing and the human body is needed to properly screen and obtain the response desired with restoration of the thoracic range of motion.

Relatedly, a manufacturer would have to have extended knowledge of the motion capture system and the electrical stimulation device to screen patients/subjects for restrictions of the thoracic spine and be able to use the machine to produce desired outcomes. This takes significant time and trial and error efforts. Hundreds of patients/subjects would need to be scanned and hundreds of hours using the electrical stimulation device would be necessary to design a protocol that allows for the restoration of thoracic range of motion.

In short, an accurate motion capture system is necessary. An electrical stimulation device that allows for elongation of muscles and muscle groups is necessary. The extensive knowledge of the human body and the mechanics of the spine are necessary. Extensive knowledge of the golf swing and how it affects the human body is also paramount.

The inventor has been able to demonstrate that thoracic range of motion deficits can be identified through 3D motion capture system and that after one 30-minute electrical-stimulation treatment, a meaningful improvement in thoracic rotation has been evidenced, as illustrated in FIG. 1. For instance, FIG. 1 illustrates that the difference between the pre-treatment data report 16 and the post-treatment data report 22 reflects a 3.9-degree left thoracic rotation increase, a 8.5-degree right thoracic rotation increase, and thus an improvement from a 68.9-degree total thoracic arc to a 81.3-degree total thoracic arc (an 18% increase), all observed on the same day. The inventor has also been able to demonstrate that the increase in the range of motion is maintained and enduring once it is obtained.

It should be understood, of course, that the foregoing relates to exemplary embodiments of the invention and that modifications may be made without departing from the spirit and scope of the invention as set forth in the following claims.

What is claimed is:

1. A method of increase a range of motion for thoracic rotation about a spine of a patient, the method comprising:
determining whether the patient has one or more thoracic range of motion deficiencies by:
obtaining said range of motion relative within a tenth of a degree; and
performing or having performed a pre-treatment report comparing a symmetry of thoracic rotation about a spine; and
if the patient has said one or more thoracic range of motion deficiencies, then externally administering an electrical stimulation for less than infinity to the patient along a sequence of muscles operatively associated with said symmetry.

2. The method of claim 1, further comprising performing or having performed a post-treatment report comparing the symmetry of thoracic rotation about the spine; and if the patient has said one or more thoracic range of motion deficiencies after the post-treatment report, then externally administering the electrical stimulation to the patient along said sequence of muscles.

3. The method of claim 1, wherein the electrical stimulation endures for less than thirty minutes.

4. The method of claim 1, wherein the pre-treatment report further compares a symmetry of lumbar rotation about said spine.

5. The method of claim 1, wherein the pre-treatment report further compares a symmetry of thoracic flexion about said spine.

6. The method of claim 1, wherein the pre-treatment report further compares a symmetry of lumbar flexion about said spine.

7. The method of claim 1, wherein the electric simulation elongates said sequence of muscles, whereby said symmetry is increased.
